(19) United States
(12) Patent Application Publication
(10) Pub. No.: US 2012/0217449 A1
(43) Pub. Date: Aug. 30, 2012

(54) MATERIALS FOR ELECTRONIC DEVICES

(75) Inventors: Hubert Spreitzer, Viernheim (DE); Jochen Schwaiger, Frankfurt Am Main (DE); Heinrich Becker, Hofheim (DE); Frank Voges, Bad Duerkheim (DE); Holger Heil, Frankfurt am Main (DE)

(73) Assignee: Merck Patent GmbH, Darmstadt (DE)

(21) Appl. No.: 13/508,263

(22) PCT Filed: Oct. 19, 2010
(86) PCT No.: PCT/EP2010/006368

§ 371 Date: May 4, 2012

(30) Foreign Application Priority Data
Nov. 6, 2009 (DE) 10 2009 053 191.2

(51) Int. Cl. H01B 1/12; C07D 403/14; C07D 213/16; C07D 401/10; C07C 2/04; C07D 471/04; C07D 251/24; C07C 49/788; C07C 13/66; C07C 211/61; C07D 239/26

(52) U.S. Cl. 252/500; 564/427; 544/180; 546/285; 544/294; 546/81; 568/326; 585/26; 585/425

(57) ABSTRACT

The present invention relates to compounds of the general formula (I), to the use thereof in electronic devices, preferably as host material for fluorescent dopants or as fluorescent dopant, to a process for the preparation of the compounds of the formula (I), and to electronic devices comprising compounds of the formula (I).

The present invention relates to compounds of the general formula (I), to the use thereof in electronic devices, to a process for the preparation of the compounds of the formula (I), and to electronic devices comprising compounds of the formula (I).

Organic semiconductors are being developed for a number of different electronic applications. The structure of organic electroluminescent devices (OLEDs) in which these organic semiconductors are employed as functional materials is described, for example, in U.S. Pat. No. 4,539,507, U.S. Pat. No. 5,151,629, EP 0676461 and WO 98/27136. However, further improvements are still desirable for use of these devices for high-quality and long-lived displays. Thus, in particular, the lifetime and efficiency of blue-emitting organic electroluminescent devices currently still represent a problem for which there is still a need for improvement. It is furthermore necessary for the compounds to have high thermal stability and a high glass-transition temperature and to be sublimable without decomposition. In particular for applications at elevated temperature, a high glass-transition temperature is essential in order to achieve long lifetimes.

For fluorescent OLEDs, mainly condensed aromatic compounds, in particular anthracene derivatives, are used in accordance with the prior art as host materials, especially for blue-emitting electroluminescent devices, for example 9,10-bis(2-naphthyl)anthracene (U.S. Pat. No. 5,935,721). WO 03/095445 and CN 1362464 disclose 9,10-bis(1-naphthyl)anthracene derivatives for use in OLEDs. Further anthracene derivatives are disclosed in WO 01/076323, in WO 01/021729, in WO 04/013073, in WO 04/018588, in WO 03/087023 or in WO 04/018587. Host materials based on aryl-substituted pyrenes and chrysenes are disclosed in WO 04/016575. Host materials based on benzanthracene derivatives are disclosed in WO 08/145,239. For high-quality applications, it is desirable to have improved host materials available.

Prior art which may be mentioned in the case of blue-emitting compounds is the use of arylvinylamines (for example WO 04/013073, WO 04/016575, WO 04/018587). However, these compounds are thermally unstable and cannot be evaporated without decomposition, which requires high technical complexity for OLED production and thus represents an industrial disadvantage. For high-quality applications, it is therefore desirable to have available improved emitters, particularly with respect to device and sublimation stability as well as emission colour.

Thus, there continues to be a demand for improved materials, in particular host materials for fluorescent and phosphorescent emitters, very particularly for blue- and green-fluorescent emitters, which are thermally stable, which result in good efficiencies and at the same time in long lifetimes in organic electronic devices, which give reproducible results during the production and operation of the device, and which are readily accessible synthetically. There continues to be a demand for fluorescent emitter materials having the above-mentioned properties. Further improvements are also necessary in the case of hole- and electron-transport materials.

The invention is based on the object of providing compounds which are particularly highly suitable for use in organic electroluminescent devices. In particular, it was an object to provide compounds by means of which an increase in the efficiency and especially the lifetime of the organic electronic device, in particular a blue-fluorescent device, is possible compared with materials in accordance with the prior art. In addition, it was a further object of the present invention to provide compounds which have high thermal stability. A further object was to provide compounds which have a lower tendency towards crystallisation during vapour deposition, causing a reduction, preferably a complete suppression, of clogging of the vapour-deposition source due to the tendency towards crystallisation or crystallisation on the target substrate or the masks.

The literature has already described individual benzo[a]anthracene derivatives which are substituted by aromatic groups (for example K. Maruyama et al., Chem. Lett. 1975, (1), 87-88; C. L. L. Chai et al., Austr. J. Chem. 1995, 48(3), 577-591, M. C. Kloetzel et al., J. Org. Chem. 1961, 26, 1748-1754 etc.). However, only the synthesis and reactivity of these compounds have been investigated. The use of these compounds in electronic devices has not been proposed. WO 2008/145239 discloses benzanthracene derivatives which are substituted in the 2-, 3-, 4-, 5- or 6-position by aromatic or heteroaromatic systems. However, linking of the said aromatic system to the anthracenyl group via a phenylene group, as described in the present application, is not disclosed. Furthermore, US 2004/0214035 has disclosed diphenylanthracene derivatives as host materials in light-emitting layers of organic electronic devices. The linking, described in the present application, of condensed polycyclic aryl or heteroaryl groups to aryl-substituted anthracene derivatives, from which compounds having particularly advantageous properties result, was, however, not disclosed in this application.

Furthermore, WO 2007/114358 discloses benzo[a]anthracene derivatives which carry an aromatic substituent in the 7-position and a hydrogen atom in the 12-position.

However, there continues to be a demand for functional materials for use in electronic devices, preferably as host materials, emitter materials, hole- or electron-transport materials in fluorescent or phosphorescent organic electroluminescent devices, which preferably have one or more of the above-mentioned advantages.

Surprisingly, it has been found that anthracene derivatives which are substituted by a six-membered aromatic ring at one of the two positions 9 and 10 and by an arylarylene or heteroarylarylene group at the other of the two positions 9 and 10 are very highly suitable for use in organic electroluminescent devices.

By means of these compounds, an increase in the efficiency and especially the lifetime of the electronic device compared with materials in accordance with the prior art is preferably possible. Furthermore, these compounds have high thermal stability. The materials are furthermore highly suitable, owing to their high glass-transition temperature, for use in electronic devices. The present invention therefore relates to these materials and to the use thereof in electronic devices, and to electronic devices comprising these materials.

The structure and numbering of the aromatic parent structures benzo[a]-anthracene, benzo[a]- and benzo[e]pyrene, benzo[c]phenanthrene, chrysene (benzo[a]phenanthrene), isochrysene (benzo[l]phenanthrene, triphenylene) and anthracene are shown below:

In order to depict that the polycyclic aromatic compounds can be bonded to further moieties of the compounds according to the invention via any of their condensed aromatic rings that are desired, a line running through all the rings in question or a line system running through all the rings in question is used in the description of this application. This is intended to be illustrated below for the example of chrysene.

The structure depicted in this case represents chrysene, which is bonded to a substituent, symbolised by *, via any desired free position, i.e. on each of its four condensed aromatic rings. Analogous representations for further compounds according to the invention are given in the following parts of this application.

The present invention relates to compounds of the formula (I)

where the following applies to the symbols and indices used:
Ar¹ is an aryl or heteroaryl group having 15 to 60 aromatic ring atoms, which may be substituted by one or more radicals R;
Ar² is an aryl or heteroaryl group having 6 to 10 aromatic ring atoms, which may be substituted by one or more radicals R¹;
Y is on each occurrence, identically or differently, CR² or N; with the proviso that not more than 2 adjacent Y simultaneously correspond to N;
X is on each occurrence, identically or differently, CR³ or N; with the proviso that not more than 2 adjacent X simultaneously correspond to N;
n is either 1, 2, 3 or 4;
R, R¹, R² are, identically or differently on each occurrence, H, D, F, Cl, Br, I, CHO, N(R⁴)₂, C(═O)R⁴, P(═O)(R⁴)₂, S(═O)R⁴, S(═O)₂R⁴, CR⁴═C(R⁴)₂, CN, NO₂, Si(R⁴)₃, B(OR⁴)₂, OSO₂R⁴, OH, a straight-chain alkyl, alkoxy or thioalkyl group having 1 to 40 C atoms or a branched or cyclic alkyl, alkoxy or thioalkyl group having 3 to 40 C atoms or an alkenyl or alkynyl group having 2 to 40 C atoms, each of which may be substituted by one or more radicals R⁴, where one or more non-adjacent CH₂ groups may be replaced by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴ and where one or more H atoms may be replaced by D, F, Cl, Br, I, CN or NO₂, or an aromatic or heteroaromatic ring system having 5 to 60 ring atoms, which may in each case be substituted by one or more non-aromatic radicals R⁴, or an aryloxy or heteroaryloxy group having 5 to 60 aromatic ring atoms, which may be substituted by one or more non-aromatic radicals R⁴, or a combination of these systems, where two or more radicals R, R¹ and/or R² may be linked to one another and may form a mono- or polycyclic, aliphatic or aromatic ring system;
R³ is, identically or differently on each occurrence, H, D, F, Cl, Br, I, CHO, N(R⁴)₂, C(═O)R⁴, P(═O)(R⁴)₂, S(═O)R⁴, S(═O)₂R⁴, CR⁴═C(R⁴)₂, CN, NO₂, Si(R⁴)₃, B(OR⁴)₂, OSO₂R⁴, OH, a straight-chain alkyl, alkoxy or thioalkyl group having 1 to 40 C atoms or a branched or cyclic alkyl, alkoxy or thioalkyl group having 3 to 40 C atoms or an alkenyl or alkynyl group having 2 to 40 C atoms, each of which may be substituted by one or more radicals R⁴, where one or more non-adjacent CH₂ groups may be replaced by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴ and where one or more H atoms may be replaced by D, F, Cl, Br, I, CN or NO₂, or a combination of these systems, where two or more radicals R³ may be linked to one another and may form a mono- or polycyclic, aliphatic ring system;
R⁴ is on each occurrence, identically or differently, H, D, F or an aliphatic, aromatic and/or heteroaromatic organic radical having 1 to 20 C atoms, in which, in addition, one or more H atoms may be replaced by F; two or more identical or different substituents R⁴ here may also be linked to one another and form a mono- or polycyclic, aliphatic or aromatic ring system,
   where, in the case where Ar¹ represents a benzo[a]anthracene derivative, this is bonded to the group Ar² in positions 1, 2, 3, 4, 5, 6, 8, 9, 10, 11 or 12.

The compounds of the formula (I) preferably have a glass-transition temperature T_{g} of greater than 70° C., particularly preferably greater than 100° C., very particularly preferably greater than 130° C.

An aryl group in the sense of this invention contains 6 to 60 C atoms; a heteroaryl group in the sense of this invention contains 1 to 59 C atoms and at least one heteroatom, with the proviso that the sum of C atoms and heteroatoms is at least 5. The heteroatoms are preferably selected from N, O and/or S. An aryl group or heteroaryl group here is taken to mean either a simple aromatic ring, i.e. benzene, or a simple heteroaromatic ring, for example pyridine, pyrimidine, thiophene, etc., or a condensed (fused) aryl or heteroaryl group, for example naphthalene, anthracene, phenanthrene, quinoline, isoquinoline, carbazole, etc.

An aryl or heteroaryl group, which may in each case be substituted by the above-mentioned radicals and which may be linked to the aromatic or heteroaromatic ring system via any desired positions, is taken to mean, in particular, groups derived from benzene, naphthalene, anthracene, phenanthrene, pyrene, dihydropyrene, chrysene, perylene, fluoranthene, benzanthracene, benzophenanthrene, tetracene, pentacene, benzopyrene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, pyrazine, phenazine, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

An aromatic ring system in the sense of this invention contains 6 to 60 C atoms in the ring system. A heteroaromatic ring system in the sense of this invention contains 1 to 59 C atoms and at least one heteroatom in the ring system, with the proviso that the sum of C atoms and heteroatoms is at least 5. The heteroatoms are preferably selected from N, O, Si, B, P and/or S. An aromatic or heteroaromatic ring system in the sense of this invention is intended to be taken to mean a system which is not necessarily formed only from an aryl or heteroaryl group, but instead in which, in addition, two or more aryl or heteroaryl groups may be connected by non-aromatic, non-conjugated units (preferably less than 10% of the atoms other than H), such as, for example, sp³-hybridised C, N, O, Si, B, P and/or S atoms, for example systems such as triarylamine or diaryl ether derivatives. It is likewise taken to mean compounds in which two or more aryl or heteroaryl groups may be connected via non-aromatic conjugated units containing sp²- or sp-hybridised C atoms or sp²-hybridised N atoms, for example systems such as stilbene, styrylnaphthalene or benzophenone derivatives. An aromatic or heteroaromatic ring system is likewise taken to mean compounds in which a plurality of aryl or heteroaryl groups are linked to one another by single bonds, for example terphenyls or diphenyltriazine. An aromatic or heteroaromatic ring system is likewise taken to mean compounds in which two or more aryl or heteroaryl groups are linked to one another by combinations of non-aromatic units and/or sp²- or sp-hybridised C atoms and/or sp²-hybridised N atoms and/or single bonds, for example systems such as 9,9′-spirobifluorene, 9,9-diarylfluorene or dihydrophenazine, phenothiazine, phenoxazine, phenoxathiine, dibenzodioxin or thianthrene derivatives.

An aromatic or heteroaromatic ring system having 5-60 ring atoms, which may also in each case be substituted by the above-mentioned radicals R⁴ and which may be linked to the aromatic or heteroaromatic group via any desired positions, is taken to mean, in particular, groups derived from benzene, naphthalene, anthracene, benzanthracene, phenanthrene, benzophenanthrene, pyrene, chrysene, perylene, fluoranthene, naphthacene, pentacene, benzopyrene, biphenyl, biphenylene, terphenyl, terphenylene, fluorene, spirobifluorene, dihydrophenanthrene, dihydropyrene, tetrahydropyrene, cis- or trans-indenofluorene, truxene, isotruxene, spirotruxene, spiroisotruxene, furan, benzofuran, isobenzofuran, dibenzofuran, thiophene, benzothiophene, isobenzothiophene, dibenzothiophene, pyrrole, indole, isoindole, carbazole, pyridine, quinoline, isoquinoline, acridine, phenanthridine, benzo-5,6-quinoline, benzo-6,7-quinoline, benzo-7,8-quinoline, phenothiazine, phenoxazine, pyrazole, indazole, imidazole, benzimidazole, naphthimidazole, phenanthrimidazole, pyridimidazole, pyrazinimidazole, quinoxalinimidazole, oxazole, benzoxazole, naphthoxazole, anthroxazole, phenanthroxazole, isoxazole, 1,2-thiazole, 1,3-thiazole, benzothiazole, pyridazine, benzopyridazine, pyrimidine, benzopyrimidine, quinoxaline, 1,5-diazaanthracene, 2,7-diazapyrene, 2,3-diazapyrene, 1,6-diazapyrene, 1,8-diazapyrene, 4,5-diazapyrene, 4,5,9,10-tetraazaperylene, pyrazine, phenazine, phenoxazine, phenothiazine, fluorubin, naphthyridine, azacarbazole, benzocarboline, phenanthroline, 1,2,3-triazole, 1,2,4-triazole, benzotriazole, 1,2,3-oxadiazole, 1,2,4-oxadiazole, 1,2,5-oxadiazole, 1,3,4-oxadiazole, 1,2,3-thiadiazole, 1,2,4-thiadiazole, 1,2,5-thiadiazole, 1,3,4-thiadiazole, 1,3,5-triazine, 1,2,4-triazine, 1,2,3-triazine, tetrazole, 1,2,4,5-tetrazine, 1,2,3,4-tetrazine, 1,2,3,5-tetrazine, purine, pteridine, indolizine and benzothiadiazole.

For the purposes of the present invention, a straight-chain alkyl group having 1 to 40 C atoms or a branched or cyclic alkyl group having 3 to 40 C atoms or an alkenyl or alkynyl group having 2 to 40 C atoms, in which, in addition, individual H atoms or CH₂ groups may be substituted by the groups mentioned above under the definition of the radicals R, is preferably taken to mean the radicals methyl, ethyl, n-propyl, i-propyl, n-butyl, i-butyl, s-butyl, t-butyl, 2-methylbutyl, n-pentyl, s-pentyl, cyclopentyl, n-hexyl, cyclohexyl, n-heptyl, cycloheptyl, n-octyl, cyclooctyl, 2-ethylhexyl, trifluoromethyl, pentafluoroethyl, 2,2,2-trifluoroethyl, ethenyl, propenyl, butenyl, pentenyl, cyclopentenyl, hexenyl, cyclohexenyl, heptenyl, cycloheptenyl, octenyl, cyclooctenyl, ethynyl, propynyl, butynyl, pentynyl, hexynyl or octynyl. An alkoxy or thioalkyl group having 1 to 40 C atoms is preferably taken to mean methoxy, trifluoromethoxy, ethoxy, n-propoxy, i-propoxy, n-butoxy, i-butoxy, s-butoxy, t-butoxy, n-pentoxy, s-pentoxy, 2-methylbutoxy, n-hexoxy, cyclohexyloxy, n-heptoxy, cycloheptyloxy, n-octyloxy, cyclooctyloxy, 2-ethylhexyloxy, pentafluoroethoxy, 2,2,2-trifluoroethoxy, methylthio, ethylthio, n-propylthio, i-propylthio, n-butylthio, i-butylthio, s-butylthio, t-butylthio, n-pentylthio, s-pentylthio, n-hexylthio, cyclohexylthio, n-heptylthio, cycloheptylthio, n-octylthio, cyclooctylthio, 2-ethylhexylthio, trifluoromethylthio, pentafluoroethylthio, 2,2,2-trifluoroethylthio, ethenylthio, propenylthio, butenylthio, pentenylthio, cyclopentenylthio, hexenylthio, cyclohexenylthio, heptenylthio, cycloheptenylthio, octenylthio, cyclooctenylthio, ethynylthio, propynylthio, butynylthio, pentynylthio, hexynylthio, heptynylthio or octynylthio.

Ar¹ preferably represents an aryl or heteroaryl group having 18 to 30 aromatic ring atoms, which may be substituted by one or more radicals R. Ar¹ particularly preferably represents an aryl group having 18 to 30 aromatic C atoms, which may be substituted by one or more radicals R. It is particularly preferred for Ar¹ to represent an angularly condensed, non-linear aryl group having 18 to 30 C atoms, which may be substituted by one or more radicals R.

For the purposes of the invention, an angularly condensed aryl group which has a non-linear structure is taken to mean an aryl group in which the aromatic rings which are condensed with one another are not connected to one another in an exclusively linear manner, i.e. via edges which are opposite one another in a parallel manner (such as, for example, in the case of naphthacene or pentacene), but instead are condensed onto one another in an angular manner at at least one position, i.e. via edges which are opposite one another in a non-parallel manner. Examples of angularly condensed, non-linear aryl groups are, inter alia, benzo[a]anthracene, chrysene and triphenylene.

Ar¹ is very particularly preferably benzo[a]anthracene, benzo[a]pyrene, benzo[e]pyrene, benzo[a]phenanthrene, benzo[c]phenanthrene or benzo-[l]phenanthrene, each of which may optionally be substituted by one or more radicals R.

In a preferred embodiment of the invention, Ar¹ represents a benzo[a]-anthracene derivative of the formula (A), which may be substituted by one or more radicals R.

The bond to the group Ar² in formula (I) here may be localised at positions 1, 2, 3, 4, 5, 6, 8, 9, 10, 11 or 12 of the benzo[a]anthracene skeleton, preferably at positions 2, 3, 4, 5 or 6. The benzo[a]anthracene group may be substituted by one or more radicals R at all free positions.

It is furthermore preferred for Ar¹ to represent a benzo[a]phenanthrene (chrysene) of the formula (B), which may be substituted by one or more radicals R.

The bond to the group Ar² in formula (I) here may be localised at positions 1, 2, 3, 4, 5, 6, 7, 8, 9, 10, 11 or 12 of the chrysene skeleton, preferably at positions 2, 6, 7, 9 or 12. The benzo[a]phenanthrene group may be substituted by one or more radicals R at all free positions.

It is likewise preferred for Ar¹ to represent a benzo[c]phenanthrene of the formula (C), which may be substituted by one or more radicals R.

The bond to the group Ar² in formula (I) here may be localised at positions 1, 2, 3, 4, 5, 6, 7, 8, 9, 10, 11 or 12 of the benzo[c]phenanthrene skeleton, preferably at positions 4, 5, 6, 7 or 8. The benzo[c]phenanthrene group may be substituted by one or more radicals R at all free positions.

It is likewise preferred for Ar¹ to represent a benzo[l]phenanthrene (isochrysene, triphenylene) of the formula (D), which may be substituted by one or more radicals R.

The bond to the group Ar² in formula (I) here may be localised at positions 1, 2, 3, 4, 5, 6, 7, 8, 9, 10, 11 or 12 of the benzo[l]phenanthrene skeleton, preferably at positions 1, 2, 3, 6 or 10. The benzo[l]phenanthrene group may be substituted by one or more radicals R at all free positions.

In an alternative preferred embodiment, Ar¹ represents a benzo[a]pyrene of the formula (E), which may be substituted by one or more radicals R.

The bond to the group Ar² in formula (I) here may be localised at positions 1, 2, 3, 4, 5, 6, 7, 8, 9, 10, 11 or 12 of the benzo[a]pyrene skeleton, preferably at positions 1, 2, 3, 6 or 12. The benzopyrene group may be substituted by one or more radicals R at all free positions.

In a further alternative preferred embodiment, Ar¹ represents a benzo[e]-pyrene of the formula (F), which may be substituted by one or more radicals R.

The bond to the group Ar² in formula (I) here may be localised at positions 1, 2, 3, 4, 5, 6, 7, 8, 9, 10, 11 or 12 of the benzo[e]pyrene skeleton, preferably at positions 2, 3, 4, 6 or 10. The benzo[e]pyrene group may be substituted by one or more radicals R at all free positions.

Ar² furthermore preferably represents an aryl group having 6 to 10 aromatic ring atoms, which is optionally substituted by one or more radicals R¹. In an alternative preferred embodiment, Ar² represents a heteroaryl group having 6 to 10 aromatic ring atoms, which is optionally substituted by one or more radicals R¹.

Ar² is particularly preferably phenylene, naphthylene, pyridinylene, pyrimidinylene, pyrazinylene, pyridazinylene, triazinylene or quinoline or isoquinoline, each of which may be substituted by one or more radicals R¹, very particularly preferably phenylene, pyridinylene, pyrimidinylene or triazinylene.

It is preferred in accordance with the invention for Ar² to represent one of the groups 1,3-phenylene, 1,4-phenylene, 1,4-naphthylene, 1,5-naphthylene, 2,6-naphthylene, 2,5-pyridinylene, 2,6-pyridinylene, 2,4-pyrimidinylene, 2,5-pyrimidinylene, 2,5-pyrazinylene, 2,4-triazinylene, 2,4-pyridazinylene, 2,5-pyridazinylene, 5,8-quinolinylene, 2,5-quinolinylene and 5,8-isoquinolinylene, each of which may be substituted by one or more radicals R¹. The two bonds which emanate from each of the groups denote the bonds to the group Ar¹ and to the central anthracene derivative in formula (I).

It is preferred in accordance with the invention for n to be equal to 1 or 2, particularly preferably equal to 1.

It is preferred for 0, 1 or 2 groups Y in the compounds of the formula (I) to be equal to N and for the remaining groups Y to be equal to CR². It is particularly preferred for all groups Y to be equal to CR².

It is preferred for radicals R² which are not equal to hydrogen to be bonded to the anthracene skeleton in the 2- or 6-position or in the 2- and 6-positions.

It is furthermore preferred for 0, 1, 2 or 3 groups X to be equal to N and for the remaining groups X to be equal to CR³. It is furthermore particularly preferred for all groups X to be equal to CR³, particularly preferably equal to CD or CH.

The radical R is preferably on each occurrence, identically or differently, H, D, F, CN, N(R⁴)₂, Si(R⁴)₃ or a straight-chain alkyl or alkoxy group having 1 to 20 C atoms or a branched or cyclic alkyl or alkoxy group having 3 to 20 C atoms, each of which may be substituted by one or more radicals R⁴, where one or more adjacent or non-adjacent CH₂ groups in the above-mentioned groups may be replaced by —C≡C—, —R⁴C═CR⁴—, Si(R⁴)₂, C═O, C═NR⁴, NR⁴, O, S, COO or CONR⁴, or an aromatic or heteroaromatic ring system having 5 to 30 ring atoms, which may in each case be substituted by one or more radicals R⁴.

The radical R¹ is preferably on each occurrence, identically or differently, H, D, F, CN, N(R⁴)₂, Si(R⁴)₃ or a straight-chain alkyl or alkoxy group having 1 to 20 C atoms or a branched or cyclic alkyl or alkoxy group having 3 to 20 C atoms, each of which may be substituted by one or more radicals R⁴, where one or more adjacent or non-adjacent CH₂ groups in the above-mentioned groups may be replaced by —C≡C—, —R⁴C═CR⁴—, Si(R⁴)₂, C═O, C═NR⁴, NR⁴, O, S, COO or CONR⁴, or an aromatic or heteroaromatic ring system having 5 to 30 ring atoms, which may in each case be substituted by one or more radicals R⁴.

The radical R² is preferably on each occurrence, identically or differently, H, D, F, CN, N(R⁴)₂, Si(R⁴)₃ or a straight-chain alkyl or alkoxy group having 1 to 20 C atoms or a branched or cyclic alkyl or alkoxy group having 3 to 20 C atoms, each of which may be substituted by one or more radicals R⁴, where one or more adjacent or non-adjacent CH₂ groups in the above-mentioned groups may be replaced by —C≡C—, —R⁴C═CR⁴—, Si(R⁴)₂, C═O, C═NR⁴, NR⁴, O, S, COO or CONR⁴, or an aromatic or heteroaromatic ring system having 5 to 30 ring atoms, which may in each case be substituted by one or more radicals R⁴.

The radical R³ is preferably on each occurrence, identically or differently, H, D, F, CN, N(R⁴)₂, Si(R⁴)₃ or a straight-chain alkyl or alkoxy group having 1 to 20 C atoms or a branched or cyclic alkyl or alkoxy group having 3 to 20 C atoms, each of which may be substituted by one or more radicals R⁴, where one or more adjacent or non-adjacent CH₂ groups in the above-mentioned groups may be replaced by —C≡C—, —R⁴C═CR⁴—, Si(R⁴)₂, C═O, C═NR⁴, NR⁴, O, S, COO or CONR⁴.

It is particularly preferred for the said preferred embodiments relating to the individual substituents R to R³ to occur together.

In a preferred embodiment of the invention, the compounds according to the invention conform to one of the formulae (I-1a) to (I-6b):

where the symbols and indices occurring are as defined above, and
Z is on each occurrence, identically or differently, CR¹ or N.

In the formulae mentioned above and in all the following formulae, it is furthermore preferred for a maximum of three groups Z per aromatic six-membered ring to be equal to N and for the remaining groups to be equal to CR¹. It is particularly preferred for all groups Z to be equal to CR¹.

The benzo[a]anthracene group in the formulae (I-1a) and (I-1b) may be bonded to the radical of the compound via positions 1, 2, 3, 4, 5, 6, 8, 9, 10, 11 or 12, preferably via positions 2, 3, 4, 5 or 6. All free positions of the benzanthracene ring may, as depicted in the corresponding formulae, optionally be substituted by a radical R.

The benzo[a]pyrene group in the formulae (I-2a) and (I-2b) may be bonded to the radical of the compound via positions 1, 2, 3, 4, 5, 6, 7, 8, 9, 10, 11 or 12, preferably via positions 1, 2, 3, 6 or 12. All free positions of the benzo[a]pyrene ring may, as depicted in the corresponding formulae, option ally be substituted by a radical R.

The benzo[e]pyrene group in the formulae (I-3a) and (I-3b) may be bonded to the radical of the compound via positions 1, 2, 3, 4, 5, 6, 7, 8, 9, 10, 11 or 12, preferably via positions 2, 3, 4, 6 or 10. All free positions of the benzo[e]pyrene ring may, as depicted in the corresponding formulae, optionally be substituted by a radical R.

The benzo[c]phenanthrene group in the formulae (I-4-a) and (I-4-b) may be bonded to the radical of the compound via positions 1, 2, 3, 4, 5, 6, 7, 8, 9, 10, 11 or 12, preferably via positions 4, 5, 6, 7 or 8. All free positions of the benzo[c]phenanthrene ring may, as depicted in the corresponding formulae, optionally be substituted by a radical R.

The benzo[a]phenanthrene group in the formulae (I-5a) and (I-5b) may be bonded to the radical of the compound via positions 1, 2, 3, 4, 5, 6, 7, 8, 9, 10, 11 or 12, preferably via positions 2, 6, 7, 9 or 12. All free positions of the chrysene ring may, as depicted in the corresponding formulae, optionally be substituted by a radical R.

The benzo[l]phenanthrene group in the formulae (I-6a) and (I-6b) may be bonded to the radical of the compound via positions 1, 2, 3, 4, 5, 6, 7, 8, 9, 10, 11 or 12, preferably via positions 1, 2, 3, 6 or 10. All free positions of the isochrysene ring may, as depicted in the corresponding formulae, optionally be substituted by a radical R.

It is furthermore preferred for the formulae (I-1a) to (I-6b) for n to be equal to 1 or 2, particularly preferably equal to 1.

In addition, the preferred embodiments mentioned for formula (I) also apply. It is particularly preferred for X to be equal to CH, CD or N.

Particularly preferred embodiments of the compounds according to the invention conform to the following formulae:

For compounds of the preferred formulae mentioned above, the preferred embodiments of the groups R, R¹, R², R³ and X, Y and Z mentioned above preferably apply. Particularly preferably, in compounds of the preferred formulae mentioned above, the group R is, identically or differently on each occurrence, H or D, Z is, identically or differently on each occurrence, CH, N or CD, Y is, identically or differently on each occurrence, CH or CD, and X is, identically or differently on each occurrence, CH, N or CD.

Examples of particularly preferred embodiments of the compounds according to the invention are the structures shown below.

The compounds of the formula (I) can be prepared by synthetic methods which are generally known to the person skilled in the art.

The compounds according to the invention can be prepared by various synthetic routes. Preferred routes will be presented below, but the person skilled in the art can, if it appears advantageous to him for the synthesis of the compounds according to the invention, deviate from these synthetic routes without being inventive and use other synthetic methods known to him for the preparation of the compounds according to the invention.

The starting compound employed can be, for example, a substituted 10-arylanthracene derivative of the formula (Z-1)

where A represents any desired reactive group and is preferably selected from I, Br, Cl, F, O-tosylates, O-triflates, O-sulfonates, boronic acid, boronic acid esters, partially fluorinated silyl groups, diazonium groups and organotin compounds.

Further important starting compounds are the benzo[a]anthracene derivatives which are halogenated, in particular brominated, in the positions according to the invention. However, the syntheses according to the invention are not restricted to the preparation of benzo[a]anthracene derivatives, but instead also encompass syntheses of other compounds of the formula (I) containing groups Ar¹ deviating from benzo[a]anthracene. Corresponding other halogenated groups Ar¹ are employed for this purpose.

Examples of the synthesis of the corresponding brominated benzo[a]-anthracene derivatives are known from the literature (2- and 3-bromobenzo[a]anthracene: Hallmark et al., J. Lab. Comp. Radiopharm. 1981, 18(3), 331; 4-bromobenzo[a]anthracene: Badgar et al., J. Chem. Soc. 1949, 799; 5-bromobenzo[a]anthracene: Newman et al., J. Org. Chem. 1982, 47(15), 2837).

Substituted or unsubstituted 5-bromobenzo[a]anthracene can alternatively also be obtained from 2-bromobenzaldehyde and 1-chloromethylnaphthalene in accordance with Scheme 1. R in Scheme 1 stands for one or more radicals, as defined for formula (I). Instead of lithiation, the reaction with another reactive metal, for example magnesium, can also be carried out in the first step. The Suzuki coupling in the first step is carried out under standard conditions, as known to the person skilled in the art of organic chemistry, for example using Pd(PPh₃)₄ in toluene/water with addition of a base at elevated temperature. The bromination in the second step can be carried out, for example, using elemental bromine or using NBS. The ring closure in the third step can be carried out, for example, by the action of polyphosphoric acid.

6-substituted benzo[a]anthracene can be obtained by firstly coupling naphthalene-2-boronic acid to 2-bromophenylacetylene (Scheme 2). The resultant acetylene can either be reacted directly in a ring-closure reaction, or it can be cyclised after halogenation, or it can be reacted with an aromatic compound in a Sonogashira coupling and subsequently cyclised. The ring closure of the acetylene is in each case carried out using an electrophile. The compounds in Scheme 2 may also be substituted by one or more radicals R, where R has the same meaning as described above under formula (I). Ar denotes an aromatic or heteroaromatic ring system. The Suzuki couplings and the Sonogashira coupling are carried out under standard conditions, as known to the person skilled in the art of organic synthesis. Preferred electrophiles for the ring-closure reaction are strong acids, such as CF₃COOH, indium halides, such as InCl₃ or InBr₃, platinum halides, such as PtCl₂, or interhalogen compounds, such as I—Cl.

The boronic acids or boronic acid derivatives derived from the compounds shown can be obtained, as shown in Scheme 3, by transmetallation, for example using n-butyllithium in THF at −78° C., and subsequent reaction of the lithiobenzo[a]anthracene formed as an intermediate with trimethyl borate, optionally followed by esterification. Furthermore, the lithiated compounds can be converted into ketones by reaction with electrophiles, such as benzonitrile, and subsequent acidic hydrolysis or into phosphine oxides by reaction with chlorodiarylphosphines and subsequent oxidation. Reaction of the lithiated compound with other electrophiles is also possible.

Suzuki coupling of the benzo[a]anthracenylboronic acids to suitable haloaryl or haloheteroaryl compounds gives access to a broad class of compounds according to the invention.

Further important starting compounds, such as the corresponding halogenated chrysene and benzo[a]pyrene compounds, can be prepared as shown in the following schemes.

For the synthesis of 6-brominated chrysene derivatives, the procedure shown, for example, in Scheme 4 can be followed (J. Org. Chem. 1987, 52, 5668-5678).

The corresponding chrysene compound is reacted here with elemental Br₂ in CS₂ solution.

For the synthesis of 6-brominated benzo[a]pyrene, the procedure shown, for example, in Scheme 5 is followed (Synlett 2005, 15, 2281-2284).

The corresponding benzo[a]pyrene compound is heated under reflux here with N-bromosuccinimide in CCl₄.

For the synthesis of 1-brominated benzo[a]pyrene, the procedure shown, for example, in Scheme 6 is followed (Eur. J. Org. Chem. 2003, 16, 3162-3166).

The 1-amino compound is prepared here in a sequence comprising chlorination, nitration and subsequent reduction. This can be converted into the desired 1-bromobenzo[a]pyrene in a further step by diazotisation and Sandmeyer reaction.

For the synthesis of 3-brominated benzo[a]pyrene, the procedure shown, for example, in Scheme 7 is followed (Chem. Pharm. Bull. 1990, 38, 3158-3161).

The corresponding benzo[a]pyrene derivative is firstly selectively nitrated here in the 3-position, then reduced, and finally the resultant amine is converted into the diazonium compound. This can then be reacted in a Sandmeyer reaction to give the desired 3-bromo compound.

The invention relates to the synthesis of compounds of the formula (I) starting from compounds of the formula (Z-2)

Ar¹-A formula (Z-2)

where Ar¹ and A are defined as indicated above, where these compounds are reacted in an organometallic coupling reaction with a derivative of the formula (Z-3)

A-Ar²-A formula (Z-3)

where Ar² and A are as defined above, and A may, on each occurrence, be identical or different and is preferably different.

A further step in the synthesis of the compounds according to the invention is an organometallic coupling reaction with a compound of the formula (Z-1) shown above.

An example of such a synthesis according to the invention is shown in Scheme 8 below.

Benzo[a]anthracenylboronic acid is reacted with bromoiodobenzene in a Suzuki coupling. The reaction here takes place selectively at the iodine atom of the benzene derivative. The product is subsequently reacted with 10-phenylanthracen-9-ylboronic acid in a second Suzuki reaction to give the compound according to the invention. The reaction can be carried out analogously with other groups Ar¹ and Ar².

A further synthesis example is shown in Scheme 9 below.

Benzo[a]pyrenylboronic acid is reacted with bromoiodobenzene in a Suzuki coupling. The reaction here takes place selectively at the iodine atom of the benzene derivative. The product is subsequently reacted with 10-phenylanthracen-9-ylboronic acid in a second Suzuki reaction to give the compound according to the invention.

A further synthesis example is shown in Scheme 10 below.

Chrysenylboronic acid (benzo[a]phenanthrenylboronic acid) is reacted with bromoiodobenzene in a Suzuki coupling. The reaction here takes place selectively at the iodine atom of the benzene derivative. The product is subsequently reacted with 10-phenylanthracen-9-ylboronic acid in a sec- and Suzuki reaction to give the compound according to the invention.

A further synthesis example is shown in Scheme 11 below.

Benzo[a]pyrenylboronic acid is reacted with 2-bromo-6-iodonaphthalene or 1-bromo-5-iodonaphthalene in a Suzuki coupling. The reaction here takes place selectively at the iodine atom of the naphthalene derivative. The product is subsequently reacted with 10-phenylanthracen-9-ylboronic acid in a second Suzuki reaction to give the compound according to the invention.

A further synthesis example is shown in Scheme 12 below.

Chrysenylboronic acid (benzo[a]phenanthrenylboronic acid) is reacted with 2-bromo-6-iodonaphthalene or 1-bromo-5-iodonaphthalene in a Suzuki coupling. The reaction here takes place selectively at the iodine atom of the naphthalene derivative. The product is subsequently reacted with 10-phenylanthracen-9-ylboronic acid in a second Suzuki reaction to give the compound according to the invention.

A further synthesis example is shown in Scheme 13 below.

Chrysenylboronic acid (benzo[a]phenanthrenylboronic acid) is reacted with dichlorophenyltriazine in a first Suzuki coupling. The reaction here takes place selectively at a chlorine atom of the triazine derivative. The product is subsequently reacted with 10-phenylanthracen-9-ylboronic acid in a second Suzuki reaction to give the compound according to the invention.

The compounds according to the invention described above, in particular compounds which are substituted by reactive leaving groups, such as bromine, iodine, boronic acid or boronic acid ester, can be used as monomers for the preparation of corresponding oligomers, dendrimers or polymers. The oligomerisation or polymerisation here is preferably carried out via the halogen functionality or the boronic acid functionality.

The invention therefore furthermore relates to oligomers, polymers or dendrimers comprising one or more compounds of the formula (I), where the bond(s) to the polymer, oligomer or dendrimer may be localised at any desired positions substituted by R, R¹, R² or R³ in formula (I). Depending on the linking of the compound of the formula (I), the compound is linked in a side chain of the oligomer or polymer or in the main chain. An oligomer in the sense of this invention is taken to mean a compound which is built up from at least three monomer units. A polymer in the sense of this invention is taken to mean a compound which is built up from at least ten monomer units. The polymers, oligomers or dendrimers according to the invention may be conjugated, partially conjugated or non-conjugated. The oligomers or polymers according to the invention may be linear, branched or dendritic. In the structures linked in a linear manner, the units of the formula (I) may be linked directly to one another or linked to one another via a divalent group, for example via a substituted or unsubstituted alkylene group, via a heteroatom or via a divalent aromatic or heteroaromatic group. In branched and dendritic structures, three or more units of the formula (I) may, for example, be linked via a trivalent or polyvalent group, for example via a trivalent or polyvalent aromatic or heteroaromatic group, to give a branched or dendritic oligomer or polymer.

For the recurring units of the formula (I) in oligomers, dendrimers and polymers, the same preferences apply as described above for compounds of the formula (I).

For the preparation of the oligomers or polymers, the monomers according to the invention are homopolymerised or copolymerised with further monomers. Suitable and preferred comonomers are selected from fluorenes (for example in accordance with EP 842208 or WO 00/22026), spirobifluorenes (for example in accordance with EP 707020, EP 894107 or WO 06/061181), para-phenylenes (for example in accordance with WO 92/18552), carbazoles (for example in accordance with WO 04/070772 or WO 04/113468), thiophenes (for example in accordance with EP 1028136), dihydrophenanthrenes (for example in accordance with WO 05/014689 or WO 07/006,383), cis- and trans-indenofluorenes (for example in accordance with WO 04/041901 or WO 04/113412), ketones (for example in accordance with WO 05/040302), phenanthrenes (for example in accordance with WO 05/104264 or WO 07/017,066) or also a plurality of these units. The polymers, oligomers and dendrimers usually also contain further units, for example emitting (fluorescent or phosphorescent) units, such as, for example, vinyltriarylamines (for example in accordance with WO 07/068,325) or phosphorescent metal complexes (for example in accordance with WO 06/003000), and/or charge-transport units, in particular those based on triarylamines.

The polymers, oligomers and dendrimers according to the invention have advantageous properties, in particular long lifetimes, high efficiencies and good colour coordinates.

The polymers and oligomers according to the invention are generally prepared by polymerisation of one or more types of monomer, at least one monomer of which results in recurring units of the formula (I) in the polymer. Suitable polymerisation reactions are known to the person skilled in the art and are described in the literature. Particularly suitable and preferred polymerisation reactions which result in C—C or C—N linking are the following:

(A) SUZUKI polymerisation;
(B) YAMAMOTO polymerisation;
(C) STILLE polymerisation; and
(D) HARTWIG-BUCHWALD polymerisation.

The way in which the polymerisation can be carried out by these methods and the way in which the polymers can then be separated off from the reaction medium and purified is known to the person skilled in the art and is described in detail in the literature, for example in WO 03/048225, WO 2004/037887 and WO 2004/037887.

The present invention thus also relates to a process for the preparation of the polymers, oligomers and dendrimers according to the invention, which is characterised in that they are prepared by SUZUKI polymerisation, YAMAMOTO polymerisation, STILLE polymerisation or HARTWIG-BUCHWALD polymerisation. The dendrimers according to the invention can be prepared by processes known to the person skilled in the art or analogously thereto. Suitable processes are described in the literature, such as, for example, in Frechet, Jean M. J.; Hawker, Craig J., “Hyperbranched polyphenylene and hyperbranched polyesters: new soluble, three-dimensional, reactive polymers”, Reactive & Functional Polymers (1995), 26(1-3), 127-36; Janssen, H. M.; Meijer, E. W., “The synthesis and characterisation of dendritic molecules”, Materials Science and Technology (1999), 20 (Synthesis of Polymers), 403-458; Tomalia, Donald A., “Dendrimer molecules”, Scientific American (1995), 272(5), 62-6; WO 02/067343 A1 and WO 2005/026144 A1.

The invention also relates to formulations comprising at least one compound of the formula (I) or a polymer, oligomer or dendrimer containing at least one unit of the formula (I) and at least one solvent, preferably an organic solvent.

The compounds of the formula (I) and the oligomers, dendrimers and polymers according to the invention are suitable for use in electronic devices, in particular in organic electroluminescent devices (OLEDs). Depending on the substitution, the compounds are employed in different functions and layers.

The invention therefore furthermore relates to the use of a compound of the formula (I) or an oligomer, dendrimer or polymer according to the invention comprising a compound of the formula (I) in electronic devices, in particular in organic electroluminescent devices.

The invention still furthermore relates to electronic devices, in particular organic electroluminescent devices (OLEDs), organic integrated circuits (O-ICs), organic field-effect transistors (O-FETs), organic thin-film transistors (O-TFTs), organic light-emitting transistors (O-LETs), organic solar cells (O-SCs), organic optical detectors, organic photoreceptors, organic field-quench devices (O-FQDs), light-emitting electrochemical cells (LECs) or organic laser diodes (O-lasers) which comprise at least one compound of the formula (I) or an oligomer, dendrimer or polymer according to the invention. Particular preference is given to organic electroluminescent devices comprising at least one compound of the formula (I) or an oligomer, dendrimer or polymer according to the invention.

The organic electroluminescent devices preferably comprise an anode, a cathode and at least one emitting layer, characterised in that at least one organic layer, which may be an emitting layer or another layer, comprises at least one compound of the formula (I) or at least one oligomer, dendrimer or polymer according to the invention. In a further preferred embodiment of the invention, the organic electroluminescent device comprises a plurality of different compounds according to the invention or oligomers, dendrimers or polymers according to the invention, which may be located together in the same layer or in different layers.

Apart from the cathode, anode and emitting layer, the organic electroluminescent device may also comprise further layers. These are selected, for example, from in each case one or more hole-injection layers, hole-transport layers, electron-blocking layers, electron-transport layers, electron-injection layers, charge-generation layers (IDMC 2003, Taiwan; Session 21 OLED (5), T. Matsumoto, T. Nakada, J. Endo, K. Mori, N. Kawamura, A. Yokoi, J. Kido, Multiphoton Organic EL Device Having Charge Generation Layer) and/or organic or inorganic p/n junctions. In addition, interlayers may also be present between the individual layers. However, it should be pointed out that each of these layers does not necessarily have to be present.

The person skilled in the art of organic electroluminescence knows which materials he can employ for these further layers. In general, all materials as used in accordance with the prior art are suitable for the further layers, and the person skilled in the art will be able to combine these materials with the materials according to the invention in an organic electroluminescent device without carrying out an inventive step.

In a further preferred embodiment of the invention, the organic electroluminescent device comprises a plurality of emitting layers, where at least one organic layer comprises at least one compound of the formula (I) or an oligomer, dendrimer or polymer according to the invention. These emission layers particularly preferably have in total a plurality of emission maxima between 380 nm and 750 nm, resulting overall in white emission, i.e. various emitting compounds which are able to fluoresce or phosphoresce and which emit blue and yellow, orange or red light are used in the emitting layers. The compound of the formula (I) is preferably used here in a blue-and/or green-emitting layer. Particular preference is given to three-layer systems, i.e. systems having three emitting layers, where one of these layers may comprise one or more compounds of the formula (I) and where the three layers exhibit blue, green and orange or red emission (for the basic structure see, for example, WO 05/011013). Emitters which have broadband emission and thus exhibit white emission are likewise suitable for white emission. A preferred embodiment of the invention thus represents an organic electroluminescent device which comprises a plurality of emitting layers and overall emits white light, where at least one layer of the device, preferably an emitting layer, comprises at least one compound of the formula (I).

In an embodiment of the invention, the compounds of the formula (I) are employed as host material for dopants, preferably for fluorescent dopants, in particular for blue- or green-fluorescent dopants. In this case, the compound according to the invention preferably contains a plurality of condensed aryl or heteroaryl groups.

In a further embodiment of the invention, the compounds of the formula (I) are employed as co-host materials together with a further host material. Their proportion in this case is preferably 5 to 95% by vol. A co-host system in the sense of the invention is a layer which comprises at least three compounds, the emitting dopant and two host materials. Further dopants and host materials may be present in the layer. The dopant here have a proportion of 0.1-30% by vol., preferably 1-20% by vol., very particularly preferably 1-10% by vol., and the two hosts together make up the remainder; the ratio of host to co-host is adjustable in a broad range, but preferably in the range from 1:10 to 10:1, particularly preferably in the range from 1:4 to 4:1.

A host material in a system comprising host and dopant is taken to mean the component which is present in the higher proportion in the system. In a system comprising one host and a plurality of dopants, the host is taken to mean the component which has the highest proportion in the mixture.

The proportion of the host material of the formula (I) in the emitting layer is between 50.0 and 99.9% by vol., preferably between 80.0 and 99.5% by vol., particularly preferably between 90.0 and 99.0% by vol. Correspondingly, the proportion of the dopant is between 0.01 and 50.0% by vol., preferably between 0.5 and 20.0% by vol. and particularly preferably between 1.0 and 10.0% by vol.

Preferred dopants are selected from the class of the monostyrylamines, the distyrylamines, the tristyrylamines, the tetrastyrylamines, the styrylphosphines, the styryl ethers and the arylamines. A monostyrylamine is taken to mean a compound which contains one substituted or unsubstituted styryl group and at least one, preferably aromatic, amine. A distyrylamine is taken to mean a compound which contains two substituted or unsubstituted styryl groups and at least one, preferably aromatic, amine. A tristyrylamine is taken to mean a compound which contains three substituted or unsubstituted styryl groups and at least one, preferably aromatic, amine. A tetrastyrylamine is taken to mean a compound which contains four substituted or unsubstituted styryl groups and at least one, preferably aromatic, amine. The styryl groups are particularly preferably stilbenes, which may also be further substituted. Corresponding phosphines and ethers are defined analogously to the amines. For the purposes of this invention, an arylamine or an aromatic amine is taken to mean a compound which contains three substituted or unsubstituted aromatic or heteroaromatic ring systems bonded directly to the nitrogen. At least one of these aromatic or heteroaromatic ring systems is preferably a condensed ring system, particularly preferably having at least 14 aromatic ring atoms. Preferred examples thereof are aromatic anthracenamines, aromatic anthracenediamines, aromatic pyrenamines, aromatic pyrenediamines, aromatic chrysenamines or aromatic chrysenediamines. An aromatic anthracenamine is taken to mean a compound in which one diarylamino group is bonded directly to an anthracene group, preferably in the 9-position. An aromatic anthracenediamine is taken to mean a compound in which two diarylamino groups are bonded directly to an anthracene group, preferably in the 9,10-position. Aromatic pyrenamines, pyrenediamines, chrysenamines and chrysenediamines are defined analogously thereto, where the diarylamino groups are preferably bonded to the pyrene in the 1-position or in the 1,6-position. Further preferred dopants are selected from indenofluorenamines or indenofluorenediamines, for example in accordance with WO 06/122630, benzoindenofluorenamines or benzoindenofluorenediamines, for example in accordance with WO 08/006,449, and dibenzoindenofluorenamines or dibenzoindenofluorenediamines, for example in accordance with WO 07/140,847. Examples of dopants from the class of the styrylamines are substituted or unsubstituted tristilbenamines or the dopants described in WO 06/000388, WO 06/058737, WO 06/000389, WO 07/065,549 and WO 07/115,610. Preference is furthermore given to the condensed hydrocarbons disclosed in the unpublished application DE 102008035413.9.

In a further preferred embodiment of the invention, the compound of the formula (I) is employed as host material in combination with an anthracene compound of the following formula (II):

where furthermore:
R⁵ is on each occurrence, identically or differently, a straight-chain alkyl group having 1 to 40 C atoms or a branched or cyclic alkyl group having 3 to 40 C atoms, each of which may be substituted by one or more radicals R⁴, where one or more non-adjacent CH₂ groups may be replaced by —R⁴C═CR⁴—, —C═C—, Si(R⁴)₂, Ge(R⁴)₂, Sn(R⁴)₂, C═O, C═S, C═Se, C═NR⁴, P(═O)(R⁴), SO, SO₂, NR⁴, —O—, —S—, —COO— or —CONR⁴— and where one or more H atoms may be replaced by D, F, Cl, Br, I, CN or NO₂, or an aromatic or heteroaromatic ring system having 5 to 60 aromatic ring atoms, which may be substituted by one or more radicals R⁴; and
Ar is on each occurrence, identically or differently, an aromatic or heteroaromatic ring system having 5 to 60 aromatic ring atoms, which may be substituted by one or more radicals R⁴, where two radicals Ar which are bonded to the same nitrogen atom may be linked to one another by a single bond or a bridge selected from B(R⁴), C(R⁴)₂, Si(R⁴)₂, C═O, C═NR⁴, C═C(R⁴)₂, O, S, S═O, SO₂, N(R⁴), P(R⁴) and P(═O)R⁴; and
R⁴ is as defined above.

In a preferred embodiment of the invention, the compound of the formula (II) is employed as fluorescent emitter compound.

Particularly preferred embodiments of the compounds of the formula (II) are shown in the following table.

Further suitable dopants for combination with a compound of the formula (I) as matrix material are the compounds depicted in the following table, and the derivatives disclosed in JP 06/001973, WO 04/047499, WO 06/098080, WO 07/065,678, US 2005/0260442 and WO 04/092111.

In a further embodiment of the invention, the compounds of the formula (I) are employed as emitting materials or within co-host systems (see above) in an emitting layer. The compounds are particularly suitable as emitting compounds if they contain at least one diarylamino unit. The compounds according to the invention are in this case particularly preferably used as green or blue emitters. The materials according to the invention are suitable as co-host if they satisfy the above-mentioned requirements as host.

The proportion of the compound of the formula (I) as dopant in the mixture of the emitting layer is in this case between 0.1 and 50.0% by vol., preferably between 0.5 and 20.0% by vol., particularly preferably between 1.0 and 10.0% by vol. The proportion of the host material is correspondingly between 50.0 and 99.9% by vol., preferably between 80.0 and 99.5% by vol., particularly preferably between 90.0 and 99.0% by vol.

The proportion of the materials according to the invention as co-host is described above.

Suitable further host materials, besides compounds of the formula (I), are materials from various classes of substance. Preferred host materials are selected from the classes of the oligoarylenes (for example 2,2′,7,7′-tetraphenylspirobifluorene in accordance with EP 676461 or dinaphthylanthracene), in particular the oligoarylenes containing condensed aromatic groups, the oligoarylenevinylenes (for example DPVBi or spiro-DPVBi in accordance with EP 676461), the polypodal metal complexes (for example in accordance with WO 04/081017), the hole-conducting compounds (for example in accordance with WO 04/058911), the electron-conducting compounds, in particular ketones, phosphine oxides, sulfoxides, etc. (for example in accordance with WO 05/084081 and WO 05/084082), the atropisomers (for example in accordance with WO 06/048268), the boronic acid derivatives (for example in accordance with WO 06/117052) or the benzanthracenes (for example in accordance with WO 08/145,239). Suitable host materials are furthermore also the compounds according to the invention. Particularly preferred host materials, apart from the compounds according to the invention, are selected from the classes of the oligoarylenes containing naphthalene, anthracene, benzanthracene and/or pyrene, or atropisomers of these compounds, the oligoarylenevinylenes, the ketones, the phosphine oxides and the sulfoxides. Very particularly preferred host materials, apart from the compounds according to the invention, are selected from the classes of the oligoarylenes containing anthracene, benzanthracene, benzophenanthrene and/or pyrene, or atropisomers of these compounds. An oligoarylene in the sense of this invention is intended to be taken to mean a compound in which at least three aryl or arylene groups are bonded to one another.

Suitable host materials are furthermore, for example, the materials depicted in the following table, and derivatives of these materials, as disclosed in WO 04/018587, WO 08/006,449, U.S. Pat. No. 5,935,721, US 2005/0181232, JP 2000/273056, EP 681019, US 2004/0247937 and US 2005/0211958.

In still a further embodiment of the invention, the compounds of the formula (I) are employed as hole-transport material in a hole-transport layer, particularly preferably as co-hole-transport material in a proportion of 5 to 95% by vol. in a hole-transport layer. The compounds are in this case preferably substituted by at least one N(Ar)₂ group. In a further preferred embodiment, the compounds of the formula (I) are employed as hole-injection material in a hole-injection layer. A hole-injection layer in the sense of this invention is a layer which is directly adjacent to the anode. A hole-transport layer in the sense of this invention is a layer which is located between a hole-injection layer and an emission layer. If the compounds of the formula (I) are used as hole-transport or hole-injection material, it may be preferred for them to be doped with electron-acceptor compounds, for example with F₄-TCNQ or with compounds as described in EP 1476881 or EP 1596445.

The hole-transport layer in the electronic devices according to the invention is optionally doped with p-dopants or undoped.

In still a further embodiment of the invention, the compounds of the formula (I) are employed as electron-transport material. It is preferred here for the compounds according to the invention to be substituted by at least one C═O, P(═O) and/or SO₂ unit. It is likewise preferred in this case for the compounds to contain one or more electron-deficient heteroaryl groups, such as, for example, imidazole, pyrazole, thiazole, benzimidazole, pyridine, pyrimidine, pyrazine, pyridazine, triazine, benzothiazole, triazole, oxadiazole, benzothiadiazole, phenanthroline, etc. It may furthermore be preferred for the compound to be doped with electron-donor compounds.

Recurring units of the formula (I) can also be employed in polymers either as polymer backbone, as emitting unit, as hole-transporting unit and/or as electron-transporting unit. The preferred substitution patterns here correspond to those described above.

Suitable charge-transport materials, as can be used in the hole-injection or hole-transport layer or in the electron-transport layer of the organic electroluminescent device according to the invention, apart from the materials according to the invention, are, for example, the compounds disclosed in Y. Shirota et al., Chem. Rev. 2007, 107(4), 953-1010, or other materials as employed in accordance with the prior art in these layers.

Examples of preferred hole-transport materials which can be used in a hole-transport or hole-injection layer in the electroluminescent device according to the invention are indenofluorenamines and derivatives (for example in accordance with WO 06/122630 or WO 06/100896), the amine derivatives disclosed in EP 1661888, hexaazatriphenylene derivatives (for example in accordance with WO 01/049806), amine derivatives containing condensed aromatic ring systems (for example in accordance with U.S. Pat. No. 5,061,569), the amine derivatives disclosed in WO 95/09147, monobenzoindenofluorenamines (for example in accordance with WO 08/006,449) or dibenzoindenofluorenamines (for example in accordance with WO 07/140,847). Hole-transport and hole-injection materials which are furthermore suitable are derivatives of the compounds depicted above, as disclosed in JP 2001/226331, EP 676461, EP 650955, WO 01/049806, U.S. Pat. No. 4,780,536, WO 98/30071, EP 891121, EP 1661888, JP 2006/253445, EP 650955, WO 06/073054 and U.S. Pat. No. 5,061,569.

Suitable hole-transport or hole-injection materials are furthermore, for example, the materials shown in the following table.

Suitable electron-transport or electron-injection materials which can be used in the electroluminescent device according to the invention are, for example, the materials shown in the following table. Suitable electron-transport and electron-injection materials are furthermore, for example, AlQ₃, BAIQ, LiQ and LiF.

The cathode of the organic electroluminescent device preferably comprises metals having a low work function, metal alloys or multilayered structures comprising various metals, such as, for example, alkaline-earth metals, alkali metals, main-group metals or lanthanoids (for example Ca, Ba, Mg, Al, In, Mg, Yb, Sm, etc.). Also suitable are alloys comprising an alkali metal or alkaline-earth metal and silver, for example an alloy comprising magnesium and silver. In the case of multilayered structures, further metals which have a relatively high work function, such as, for example, Ag or Al, can also be used in addition to the said metals, in which case combinations of the metals, such as, for example, Ca/Ag, Ba/Ag or Mg/Ag, are then generally used. It may also be preferred to introduce a thin interlayer of a material having a high dielectric constant between a metallic cathode and the organic semiconductor. Suitable for this purpose are, for example, alkali metal fluorides or alkaline-earth metal fluorides, but also the corresponding oxides or carbonates (for example LiF, Li₂O, BaF₂, MgO, NaF, CsF, Cs₂CO₃, etc.). Furthermore, lithium quinolinate (LiQ) can be used for this purpose. The layer thickness of this layer is preferably between 0.5 and 5 nm.

The anode preferably comprises materials having a high work function. The anode preferably has a work function of greater than 4.5 eV vs. vacuum. Suitable for this purpose are on the one hand metals having a high redox potential, such as, for example, Ag, Pt or Au. On the other hand, metal/metal oxide electrodes (for example Al/Ni/NiOₓ, Al/PtOₓ) may also be preferred. For some applications, at least one of the electrodes must be transparent or partially transparent in order to facilitate either irradiation of the organic material (organic solar cell) or the coupling-out of light (OLEDs, O-lasers). Preferred anode materials here are conductive mixed metal oxides. Particular preference is given to indium tin oxide (ITO) or indium zinc oxide (IZO). Preference is furthermore given to conductive, doped organic materials, in particular conductive, doped polymers.

The device is appropriately (depending on the application) structured, provided with contacts and finally sealed, since the lifetime of the devices according to the invention is shortened in the presence of water and/or air.

Preference is furthermore given to an organic electroluminescent device, characterised in that one or more layers are applied by means of a sublimation process, in which the materials are applied by vapour deposition in vacuum sublimation units at an initial pressure of less than 10⁻⁵ mbar, preferably less than 10⁻⁶ mbar. However, it is also possible here for the initial pressure to be even lower, for example less than 10⁻⁷ mbar.

Preference is likewise given to an organic electroluminescent device, characterised in that one or more layers are applied by means of the OVPD (organic vapour phase deposition) process or with the aid of carrier-gas sublimation, in which the materials are applied at a pressure of between 10⁻⁵ mbar and 1 bar. A special case of this process is the OVJP (organic vapour jet printing) process, in which the materials are applied directly through a nozzle and are thus structured (for example M. S. Arnold et al., Appl. Phys. Lett. 2008, 92, 053301).

Preference is furthermore given to an organic electroluminescent device, characterised in that one or more layers are produced from solution, such as, for example, by spin coating, or by means of any desired printing process, such as, for example, screen printing, flexographic printing, nozzle printing or offset printing, but particularly preferably LITI (light induced thermal imaging, thermal transfer printing) or ink-jet printing. Soluble compounds are necessary for this purpose. High solubility can be achieved through suitable substitution of the compounds.

The present application text is directed to the use of the compounds according to the invention in OLEDs and in corresponding displays. However, it is possible for the person skilled in the art, without further inventive step, also to employ the compounds according to the invention in other electronic devices, for example in organic field-effect transistors (O-FETs), organic thin-film transistors (O-TFTs), organic light-emitting transistors (O-LETs), organic integrated circuits (O-ICs), organic solar cells (O-SCs), organic field-quench devices (O-FQDs), light-emitting electrochemical cells (LECs), organic laser diodes (O-lasers) or organic photoreceptors.

The present invention likewise relates to the use of the compounds according to the invention in the corresponding devices and to these devices themselves.

On use in organic electroluminescent devices, the compounds according to the invention preferably have high efficiency and a long lifetime, making the organic electroluminescent devices according to the invention very highly suitable for use in high-quality and long-lived displays. Furthermore, the compounds according to the invention have high thermal stability and a high glass-transition temperature and can be sublimed without decomposition. An additional advantage over the materials known from the prior art is the fact that they preferably have a lower tendency towards crystallisation during vapour deposition at the vapour-deposition source. Clogging of the vapour-deposition source during production of the electronic devices according to the invention consequently does not occur or only occurs to a slight extent, which represents an important advantage, in particular for mass production.

The following examples are intended to explain the invention in greater detail. The examples do not have a restrictive character, i.e. the invention is not restricted to the examples mentioned. The person skilled in the art will be able to prepare further compounds according to the invention and employ these in electronic devices without an inventive step.

### EXAMPLES

### Synthesis Example 1

### Preparation of 4-[3-(10-phenylanthracen-9-yl)phenyl]benzo[a]anthracene (H3)

### 1st Step:

With N₂ aeration, a 4 l flask is dried by heating, and 100 g (325.5 mmol) of 4-bromobenzo[a]anthracene are initially introduced in 1400 ml of dry THF. The batch is cooled to −72° C., and 190 ml of 2.5 M n-butyllithium are rapidly added dropwise. In the process, warming from −72° C. to −61° C. occurs (duration of addition: 2 min). The reaction mixture is stirred at −70° C. for a further 3 h.

150 ml (637 mmol) of triisopropyl borate are immediately allowed to run into the solution via a dropping funnel, during which the batch warms to −68° C. The batch is subsequently stirred at −70° C. for 2 h and then allowed to warm to RT. The reaction solution is diluted with 1300 ml of ethyl acetate and 690 ml of water in a 6 l washing flask under a stream of N₂ and stirred for 60 min. The aqueous phase is subsequently separated off, and the organic phase is washed 2× with 750 ml of water each time. The organic phase is dried using Na₂SO₄ and evaporated to 70 ml of ethyl acetate solution in a rotary evaporator.

Yield: 62.13 g (70% of theory)

### 2nd Step:

Sodium carbonate (206.2 g, 0.47 mol), boronic acid (127.8 g, 0.47 mol) and bromoiodobenzene (199.4 g, 0.7 mol) are initially introduced. 825 ml of toluene, 625 ml of water and 250 ml of ethanol are added, and the suspension is degassed for about 30 min, and tetrakistriphenylphosphinepalladium catalyst (5.773 g, 5 mmol) is then added. The reaction mixture is heated under reflux for 12 hours (oil-bath temp.: 100° C.).

Completion of the reaction is subsequently monitored by TLC (TLC solvent: heptane/EA 5:1), and the mixture is then allowed to cool. The mixture is diluted with water and toluene, the phases are separated, and the combined organic phases are washed with water and concentrated to ⅓ of the volume. The precipitated solid is filtered off.

Yield: 142.3 g (79% of theory)

### 3rd Step:

The product from step 2 (142.3 g, 0.37 mol), the boronic acid ester (155.3 g, 0.41 mol) and the potassium phosphate (165.5 g, 7.80 mol) are initially introduced in a flask, and 1000 ml of toluene, 1000 ml of water and 415 ml of dioxane are then added. The mixture is degassed for 30 minutes with stirring by passing argon through. The phosphine (6.8 g, 22.28 mmol) is then added, the mixture is stirred briefly, and the palladium(II) acetate (833 mg, 3.71 mmol) is then added. Finally, the mixture is heated under reflux (oil bath 120° C.) and refluxed for 24 hours. The mixture is then allowed to cool. Glacial acetic acid/ethanol 1:1 (1200 ml) is subsequently added. The precipitated solid is filtered off with suction, rinsed 2× with about 250 ml of toluene, 2× with about 450 ml of water/ethanol mixture (ratio 1:1) and finally 2× with 550 ml of ethanol. The solid is extracted with 3 l of toluene for 72 hours in a hot Soxhlet extractor and subsequently washed by stirring in degassed acetonitrile and degassed dichloromethane under reflux. The product is sublimed at 5×10⁻⁶ mbar and about 320° C.

Yield: 114 g (55% of theory)

### Synthesis Example 2

### Preparation of 4-[4-(10-phenylanthracen-9-yl)phenyl]benzo[a]anthracene (H2)

### 1st Step:

Sodium carbonate (250 g, 2.36 mol), benzanthraceneboronic acid (see Synthesis Example 1, step 1) (155 g, 0.57 mol) and 4-bromoiodobenzene (242.9 g, 0.86 mol) are initially introduced.

1000 ml of toluene, 750 ml of water and 300 ml of ethanol are added, and the suspension is degassed for about 30 min, and tetrakistriphenylphosphinepalladium (7 g, 6.05 mmol) is then added. The reaction mixture is heated under reflux for 15 hours with vigorous stirring (oil-bath temp.: 100° C.).

TLC monitoring (TLC solvent: heptane/EA 5:1) shows complete conversion, and the mixture is then allowed to cool. The mixture is diluted with water and toluene, the phases are separated, and the combined organic phases are washed firstly with water, then with sat. NaCl solution. The precipitated solid is filtered off.

Yield: 168.5 g (77% of theory)

### 2nd Step:

168.4 g (0.44 mol) of 4-(4-bromophenyl)benzo[a]anthracene, 183.9 g (0.48 mol) of 9-phenylanthracene-10-boronic acid pinacol ester and potassium phosphate (195.5 g, 0.92 mol) are initially introduced in a 4 l flask, and 1200 ml of toluene, 1200 ml of water and 475 ml of dioxane are then added. The mixture is degassed for 30 minutes with stirring while passing argon through. The tris-o-tolylphosphine (8.0 g, 26.4 mmol) is then added, the mixture is stirred briefly, and palladium(II) acetate (986 mg, 4.4 mmol) is then added. Finally, the mixture is heated under reflux (oil bath 120° C.) and refluxed for 39 hours. A further 18 g of boronic acid ester are added, and the mixture is heated under reflux for a further 10 h. The mixture is then allowed to cool. Glacial acetic acid/ethanol 1:1 (1500 ml) is subsequently added. The precipitated solid is filtered off with suction, rinsed 2× with about 250 ml of toluene, 2× with about 450 ml of water/ethanol mixture (ratio 1:1) and finally 2× with 550 ml of ethanol. The solid is extracted with 3 l of toluene in a hot extractor for 5 days and subsequently recrystallised 4× from degassed dioxane. The product is sublimed at 5×10⁻⁶ mbar and about 330° C.

Yield: 85 g (35% of theory)

### Synthesis Example 3

### Preparation of 5-[10-(4-benzo[a]anthracen-4-ylphenyl)anthracen-9-yl]pyrimidine (ETM2)

### 1st Step:

44.6 g (173 mmol) of bromoanthracene are dissolved in 340 ml of dry THF in a 4 l four-necked flask and cooled to −75° C., during which a brown-green suspension forms.

69.5 ml of 2.5 M n-BuLi solution in hexane are added at this temperature over the course of about 30 min, and the mixture is stirred for a further 2 h. 49.6 ml (210 mmol) of triisopropyl borate are then added dropwise at −75° C. over the course of 25 min, and the mixture is stirred for a further 2 h and warmed to room temperature overnight.

19.7 g (123.9 mmol) of bromopyrimidine, 500 ml of toluene, 195 ml of a 20% tetraethylammonium hydroxide solution in water are degassed for 30 min using N₂ in a further 4 l four-necked flask; a pale-brown, clear solution forms. 2.86 g (2.47 mmol) of Pd(PPh₃)₄ and the solution of the boronic acid are added, and the mixture is refluxed for 6 h. 300 ml of toluene and 450 ml of water are added, and the org. phase is washed 2× with water and 1× with sat. NaCl solution and dried over MgSO₄. The mixture is concentrated in a rotary evaporator, and the product is precipitated using heptane, rinsed with heptane and dried, giving 32.3 g (quant.) of 5-anthracen-9-yl-pyrimidine.

### 2nd Step:

73.8 g of 5-anthracen-9-ylpyrimidine (288 mmol) are dissolved in 800 ml of CH₂Cl₂ in a 2 l four-necked flask; the solution is degassed by passing N₂ through. 54.0 g (302 mmol) of NBS are added, and the suspension is stirred overnight at RT with exclusion of light. The reaction mixture is then evaporated to dryness in a rotary evaporator, the residue is taken up in 300 ml of ethanol, the solution is stirred at RT for 30 min., and the product is filtered off with suction, washed 1× with 300 ml of ethanol and sucked dry. It is washed by boiling in 1 l of ethanol and dried, giving 87.5 g (261 mmol, 91%) of 5-(10-bromoanthracen-9-yl)pyrimidine as yellow solid.

### 3rd Step:

67.0 g (200 mmol) of 5-(10-bromoanthracen-9-yl)pyrimidine are initially introduced in a 2000 ml four-necked flask and dissolved in 1000 ml of anhydrous diethyl ether and cooled to 0-5° C. 88 ml of 2.5 M n-BuLi are slowly added dropwise. The mixture is stirred at RT for 2 h.

The reaction mixture is then cooled to −75° C., and 29 ml (260 mmol) of trimethyl borate, diluted with 50 ml of diethyl ether, are added over the course of 1 min with stirring.

The mixture is stirred at −75° C. for 1 h and warmed to +10° C. 500 ml of water are added, the phases are separated, and the organic phase is evaporated. The solid is washed with hexane and dried, giving 55.8 g (186 mmol, 93%) of 5-(10-boronylanthracen-9-yl)pyrimidine.

### 4th Step:

4-(4-Bromophenyl)benzo[a]anthracene (59.4 g, 0.155 mol), 48.9 g (0.163 mmol) of 5-(10-boronylanthracen-9-yl)pyrimidine and potassium phosphate (65.2 g, 0.30 mol) are initially introduced in a 2 l flask, and 400 ml of toluene, 400 ml of water and 150 ml of dioxane are then added. The mixture is degassed for 30 minutes with stirring by passing argon through. Tris-o-tolylphosphine (2.8 g, 8.8 mmol) is then added, the mixture is stirred briefly, and palladium(II) acetate (330 mg, 1.45 mmol) is then added. Finally, the mixture is heated under reflux (oil bath 120° C.) and refluxed for 24 hours. The mixture is then allowed to cool. Glacial acetic acid/ethanol 1:1 (500 ml) is subsequently added. The precipitated solid is filtered off with suction, rinsed 2× with about 100 ml of toluene, 2× with about 150 ml of water/ethanol mixture (ratio 1:1) and finally 2× with 200 ml of ethanol. The solid is extracted with 1 l of toluene in a hot extractor for 5 days and subsequently recrystallised 4× from degassed o-xylene. The product is sublimed at 3×10⁻⁶ mbar and about 330° C. Yield: 37.2 g (43%).

### Synthesis Example 4

### Preparation of 5-[10-(3-benzo[a]anthracen-4-ylphenyl)anthracen-9-yl]-N,N,N′,N′-tetra-p-tolylbenzene-1,3-diamine (HTM2)

### 1st Step:

49.1 g (190 mmol) of bromoanthracene are dissolved in 380 ml of dry THF in a 4 l four-necked flask and cooled to −75° C., during which a brown-green suspension forms. 76.5 ml of 2.5 M n-BuLi solution in hexane are added at this temperature over the course of about 30 min, and the mixture is stirred for a further 2 h. 55 ml (230 mmol) of triisopropyl borate are then added dropwise at −70° C. over the course of 230 min, and the mixture is stirred for a further 2 h and warmed to room temperature overnight. 75.0 g (137 mmol) of 5-bromo-N,N,N′,N′-tetra-p-tolylbenzene-1,3-diamine (preparation analogous to EP 1969083), 600 ml of toluene, 220 ml of a 20% tetraethylammonium hydroxide solution in water are degassed for 30 min using N₂ in a further 4 l four-necked flask; a pale-brown, clear solution forms. 3.15 g (2.71 mmol) of Pd(PPh₃)₄ and the solution of the boronic acid are added, and the mixture is heated under reflux for 8 h. 400 ml of toluene and 500 ml of water are added, and the organic phase is washed 2× with water and 1× with sat. NaCl solution and dried over MgSO₄. The mixture is concentrated in a rotary evaporator, and the product is precipitated using heptane, rinsed with heptane and dried, giving 88.5 g (quant.) of 5-anthracen-9-yl-N,N,N′,N′-tetra-p-tolylbenzene-1,3-diamine.

### 2nd Step:

84.0 g (130 mmol) of 5-anthracen-9-yl-N,N,N′,N′-tetra-p-tolylbenzene-1,3-diamine are dissolved in 350 ml of CH₂Cl₂ in a 2 l flask; the solution is degassed by passing N₂ through. 24.4 g (136 mmol) of NBS are added, and the suspension is stirred overnight at RT with exclusion of light.

The reaction mixture is then evaporated to dryness in a rotary evaporator, the residue is taken up in 300 ml of ethanol, the solution is stirred at RT for 30 min, and the product is then filtered off with suction, washed 1× with 300 ml of ethanol and dried. The product is washed with 500 ml of boiling ethanol and dried, giving 94.1 g (113 mmol, 87%) of 5-(10-bromoanthracen-9-yl)-N,N,N′,N′-tetra-p-tolylbenzene-1,3-diamine as yellow solid.

### 3rd Step:

72.4 g (100 mmol) of the bromide are dissolved in 500 ml of anhydrous diethyl ether in a 2 l four-necked flask and cooled to 0-5° C. 44 ml of 2.5 M n-BuLi (110 mmol) are slowly added dropwise. The mixture is stirred at RT for 2 h.

The reaction mixture is then cooled to −75° C., and 14.5 ml (130 mmol) of trimethyl borate, diluted with 25 ml of diethyl ether, are added over the course of 1 min with stirring. The mixture is stirred at −75° C. for 1 h and warmed to +10° C. 250 ml of water are added, the phases are separated, and the organic phase is evaporated. The solid is washed with hexane and dried, giving 62.7 g (91 mmol, 91%) of 5-(10-boronylanthracen-9-yl)N,N,N′,N′-tetra-p-tolylbenzene-1,3-diamine.

### 4th Step:

4-(3-Bromophenyl)benzo[a]anthracene (32.7 g, 85 mmol, see Synthesis Example 1, 2nd step), 5-(10-boronylanthracen-9-yl)-N,N,N′,N′-tetra-p-tolylbenzene-1,3-diamine (61.7 g, 89.6 mmol) and potassium phosphate (35.9 g, 160 mmol) are initially introduced in a 1 l flask, and 200 ml of toluene, 200 ml of water and 75 ml of dioxane are then added. The mixture is degassed for 30 min with stirring by passing argon through. Tris-o-tolylphosphine (1.05 g, 4.8 mmol) is then added, the mixture is stirred briefly, and palladium(II) acetate (160 mg, 0.8 mmol) is then added. Finally, the mixture is heated under reflux (oil bath 120° C.) for 20 h. The mixture is then allowed to cool. Glacial acetic acid/ethanol 1:1 (300 ml) is subsequently added. The precipitated solid is filtered off with suction, rinsed 2× with about 100 ml of toluene, 2× with about 150 ml of water/ethanol mixture (ratio 1:1) and finally 2× with 100 ml of ethanol. The solid is extracted with 1 l of chlorobenzene in a hot extractor for 2 days and subsequently recrystallised 6× from degassed chlorobenzene. The product is sublimed at 4×10⁻⁶ mbar and about 365° C. Yield: 37.0 g (46%).

### Synthesis Examples 5 and 6

### Synthesis of compounds H5 and H6

Compounds H5 and H6 are prepared analogously to Synthesis Examples 1 and 2 (H2 and H3), but in each case 9-(1-naphthyl)anthracene-10-boronic acid is employed instead of 9-phenylanthracene-10-boronic acid in the final step.

### Device Examples: Production of OLEDs

OLEDs according to the invention and OLEDs in accordance with the prior art are produced by a general process in accordance with WO 04/058911, which is adapted to the circumstances described here (layer-thickness variation, materials used).

The results for various OLEDs are presented in the following Examples 1 to 28 (see Tables 1 and 2). Glass plates coated with structured ITO (indium tin oxide) in a thickness of 150 nm are coated with 20 nm of PEDOT (poly(3,4-ethylenedioxy-2,5-thiophene), spin-coated from water; purchased from H. C. Starck, Goslar, Germany) for improved processing. These coated glass plates form the substrates to which the OLEDs are applied. The OLEDs have in principle the following layer structure: substrate/hole-transport layer (HTL)/optional interlayer (IL)/electron-blocking layer (EBL)/emission layer (EML)/optional hole-blocking layer (HBL)/electron-transport layer (ETL)/optional electron-injection layer (EIL) and finally a cathode. The cathode is formed by an aluminium layer with a thickness of 100 nm. The precise structure of the OLEDs is shown in Table 1. The materials used for the production of the OLEDs are shown in Table 3.

All materials are applied by thermal vapour deposition in a vacuum chamber. The emission layer here always consists of at least one matrix material (host material) and an emitting dopant (emitter), which is admixed with the matrix material or materials in a certain proportion by volume by coevaporation. Information such as H1:SEB1 (95%:5%) here means that material H1 is present in the layer in a proportion by volume of 95% and SEB1 is present in a proportion by volume of 5%. Analogously, the electron-transport layer may also consist of a mixture of two materials.

The OLEDs are characterised by standard methods. For this purpose, the electroluminescence spectra, the current efficiency (measured in cd/A), the power efficiency (measured in Im/W) and the external quantum efficiency (EQE, measured in percent) as a function of the luminous density, calculated from current/voltage/luminance characteristic lines (IUL characteristic lines), and the lifetime are determined. The lifetime is defined as the time after which the luminous density has dropped from a certain initial luminous density Iₒ to a certain proportion. LD50 means that the said lifetime is the time by which the luminous density has dropped to 0.5·I₀ (to 50%), i.e. from, for example, 6000 cd/m² to 3000 cd/m².

The compounds according to the invention can be employed, inter alia, as matrix materials (host materials) for fluorescent dopants. Compounds H2 and H3 according to the invention are used here. Compounds H1, H4, H5 and H6 are used as comparison in accordance with the prior art. OLEDs comprising the blue-emitting dopant SEB1 are shown. Furthermore, results with the green-emitting dopant SEG1 are shown. The results for the OLEDs are shown in Table 2. Ex. 1-9 show OLEDs comprising materials in accordance with the prior art and serve as comparative examples. OLEDs 10-28 according to the invention show the advantages on use of compounds of the formula (I).

The use of compounds according to the invention enables improvements to be achieved in processing and material stability compared with the prior art. The electrical performance is found to be at least comparable or better than the reference.

Compared with devices comprising compounds in accordance with the prior art, the electrical characteristic data of the devices according to the invention are comparable or better in all cases. With an otherwise identical layer structure, devices using H2 or H3 exhibit longer operating lifetimes and higher power efficiency. Devices which use charge-transport materials ETM2 or HTM2 according to the invention exhibit a lower operating voltage and an increased lifetime.

TABLE 1
Structure of the OLEDs

| | HTL | IL | EBL | EML | ETL | EIL |
|---|---|---|---|---|---|---|
| Ex. | thickness | thickness | thickness | thickness | thickness | thickness |
| 1 (comp.) | HTM1 | HIL1 | NPB | H1:SEB1 | ETM1:LiQ | |
| | 140 nm | 5 nm | 20 nm | (95%:5%) | (50:50) | |
| | | | | 20 nm | 30 nm | |
| 2 (comp.) | HTM1 | HIL1 | NPB | H1:SEB1 | ETM1:LiQ | |
| | 140 nm | 5 nm | 20 nm | (95%:5%) | (25:75) | |
| | | | | 20 nm | 30 nm | |
| 3 (comp.) | HTM1 | HIL1 | NPB | H1:SEB1 | ETM1 | LiQ |
| | 140 nm | 5 nm | 20 nm | (95%:5%) | 30 nm | 3 nm |
| | | | | 20 nm | | |
| 4 (comp.) | HTM1 | HIL1 | NPB | H1:SEB1 | Alq | LiF |
| | 140 nm | 5 nm | 20 nm | (95%:5%) | 30 nm | 1 nm |
| | | | | 20 nm | | |
| 5 (comp.) | HTM1 | | NPB | H4:TER1 | Alq | LiF |
| | 20 nm | | 20 nm | (85%:15%) | 20 nm | 1 nm |
| | | | | 30 nm | | |
| 6 (comp.) | HTM1 | | EBM1 | H4:TEG1 | ETM1:LiQ | |
| | 160 nm | | 20 nm | (90%:10%) | (50%:50%) | |
| | | | | 30 nm | 40 nm | |
| 7 (comp.) | HTM1 | HIL1 | NPB | H1:SEG1 | ETM1 | LiQ |
| | 140 nm | 5 nm | 20 nm | (97%:3%) | 30 nm | 3 nm |
| | | | | 20 nm | | |
| 8 (comp.) | HTM1 | HIL1 | NPB | H5:SEB1 | ETM1 | LiQ |
| | 140 nm | 5 nm | 20 nm | (95%:5%) | 30 nm | 3 nm |
| | | | | 20 nm | | |
| 9 (comp.) | HTM1 | HIL1 | NPB | H6:SEB1 | ETM1 | LiQ |
| | 140 nm | 5 nm | 20 nm | (95%:5%) | 30 nm | 3 nm |
| | | | | 20 nm | | |
| 10 | HTM1 | HIL1 | NPB | H3:SEB1 | ETM1:LiQ | |
| | 140 nm | 5 nm | 20 nm | (95%:5%) | (50:50) | |
| | | | | 20 nm | 30 nm | |
| 11 | HTM1 | HIL1 | NPB | H3:SEB1 | ETM1:LiQ | |
| | 140 nm | 5 nm | 20 nm | (95%:5%) | (25:75) | |
| | | | | 20 nm | 30 nm | |
| 12 | HTM1 | HIL1 | NPB | H3:SEB1 | ETM1 | LiQ |
| | 140 nm | 5 nm | 20 nm | (95%:5%) | 30 nm | 3 nm |
| | | | | 20 nm | | |
| 13 | HTM1 | HIL1 | NPB | H3:SEB1 | Alq | LiF |
| | 140 nm | 5 nm | 20 nm | (95%:5%) | 30 nm | 1 nm |
| | | | | 20 nm | | |
| 14 | HTM1 | HIL1 | NPB | H2:SEB1 | ETM1:LiQ | |
| | 140 nm | 5 nm | 20 nm | (95%:5%) | (50:50) | |
| | | | | 20 nm | 30 nm | |
| 15 | HTM1 | HIL1 | NPB | H2:SEB1 | ETM1:LiQ | |
| | 140 nm | 5 nm | 20 nm | (95%:5%) | (25:75) | |
| | | | | 20 nm | 30 nm | |
| 16 | HTM1 | HIL1 | NPB | H2:SEB1 | ETM1 | LiQ |
| | 140 nm | 5 nm | 20 nm | (95%:5%) | 30 nm | 3 nm |
| | | | | 20 nm | | |
| 17 | HTM1 | HIL1 | NPB | H2:SEB1 | Alq | LiF |
| | 140 nm | 5 nm | 20 nm | (95%:5%) | 30 nm | 1 nm |
| | | | | 20 nm | | |
| 18 | HTM2 | HIL1 | NPB | H1:SEB1 | ETM1:LiQ | |
| | 140 nm | 5 nm | 20 nm | (95%:5%) | (50:50) | |
| | | | | 20 nm | 30 nm | |
| 19 | HTM2 | HIL1 | NPB | H1:SEB1 | ETM1:LiQ | |
| | 140 nm | 5 nm | 20 nm | (95%:5%) | (25:75) | |
| | | | | 20 nm | 30 nm | |
| 20 | HTM2 | HIL1 | NPB | H1:SEB1 | ETM1 | LiQ |
| | 140 nm | 5 nm | 20 nm | (95%:5%) | 30 nm | 3 nm |
| | | | | 20 nm | | |
| 21 | HTM2 | HIL1 | NPB | H1:SEB1 | Alq | LiF |
| | 140 nm | 5 nm | 20 nm | (95%:5%) | 30 nm | 1 nm |
| | | | | 20 nm | | |
| 22 | HTM1 | HIL1 | NPB | H1:SEB1 | ETM2:LiQ | |
| | 140 nm | 5 nm | 20 nm | (95%:5%) | (50:50) | |
| | | | | 20 nm | 30 nm | |
| 23 | HTM1 | HIL1 | NPB | H1:SEB1 | ETM2:LiQ | |
| | 140 nm | 5 nm | 20 nm | (95%:5%) | (25:75) | |
| | | | | 20 nm | 30 nm | |
| 24 | HTM1 | HIL1 | NPB | H1:SEB1 | ETM2 | LiQ |
| | 140 nm | 5 nm | 20 nm | (95%:5%) | 30 nm | 3 nm |
| | | | | 20 nm | | |
| 25 | HTM2 | | NPB | H4:TER1 | Alq | LiF |
| | 20 nm | | 20 nm | (85%:15%) | 20 nm | 1 nm |
| | | | | 30 nm | | |
| 26 | HTM2 | | EBM1 | H4:TEG1 | ETM1:LiQ | |
| | 160 nm | | 20 nm | (90%:10%) | (50%:50%) | |
| | | | | 30 nm | 40 nm | |
| 27 | HTM1 | | EBM1 | H4:TEG1 | ETM2:LiQ | |
| | 160 nm | | 20 nm | (90%:10%) | (50%:50%) | |
| | | | | 30 nm | 40 nm | |
| 28 | HTM1 | HIL1 | NPB | H2:SEG1 | ETM1 | LiQ |
| | 140 nm | 5 nm | 20 nm | (97%:3%) | 30 nm | 3 nm |
| | | | | 20 nm | | |

TABLE 2
Results for the OLEDs

| | Voltage [V] | Efficiency | Efficiency | | | |
|---|---|---|---|---|---|---|
| | for | [cd/A] at | [lm/W] at | CIE x/y at | | LD50 |
| Ex. | 1000 cd/m² | 1000 cd/m² | 1000 cd/m² | 1000 cd/m² | | I = 6000 cd/m² |
| 1 (comp.) | 4.2 | 9.6 | 7.3 | 0.142 | 0.145 | 180 |
| 2 (comp.) | 5.1 | 7.3 | 4.5 | 0.142 | 0.147 | 490 |
| 3 (comp.) | 3.6 | 7.9 | 6.8 | 0.142 | 0.150 | 80 |
| 4 (comp.) | 5.7 | 5.4 | 3.0 | 0.149 | 0.169 | 240 |
| 5 (comp.) | 4.7 | 7.1 | 4.7 | 0.69 | 0.31 | 420 |
| 6 (comp.) | 4.6 | 54 | 37 | 0.37 | 0.60 | 400* |
| 7 (comp.) | 3.5 | 20 | 18 | 0.26 | 0.67 | 310** |
| 8 (comp.) | 4.3 | 6.4 | 4.7 | 0.143 | 0.148 | 85 |
| 9 (comp.) | 4.4 | 7.8 | 5.5 | 0.143 | 0.146 | 135 |
| 10 | 4.1 | 9.9 | 7.6 | 0.142 | 0.145 | 270 |
| 11 | 4.7 | 7.6 | 5.0 | 0.142 | 0.146 | 510 |
| 12 | 3.6 | 7.9 | 6.9 | 0.143 | 0.147 | 170 |
| 13 | 5.6 | 5.3 | 3.9 | 0.148 | 0.163 | 420 |
| 14 | 4.3 | 8.1 | 6.0 | 0.143 | 0.141 | 280 |
| 15 | 4.9 | 7.4 | 4.8 | 0.143 | 0.142 | 560 |
| 16 | 3.9 | 6.3 | 5.1 | 0.143 | 0.147 | 160 |
| 17 | 6.1 | 5.5 | 2.8 | 0.151 | 0.168 | 260 |
| 18 | 3.9 | 9.6 | 7.7 | 0.142 | 0.145 | 190 |
| 19 | 4.5 | 7.4 | 5.2 | 0.142 | 0.146 | 500 |
| 20 | 3.3 | 7.8 | 7.5 | 0.142 | 0.150 | 210 |
| 21 | 5.0 | 5.5 | 3.5 | 0.149 | 0.169 | 280 |
| 22 | 3.5 | 9.2 | 8.2 | 0.142 | 0.146 | 270 |
| 23 | 4.4 | 7.3 | 5.2 | 0.142 | 0.147 | 590 |
| 24 | 3.5 | 7.9 | 7.1 | 0.142 | 0.149 | 220 |
| 25 | 4.3 | 8.1 | 5.9 | 0.69 | 0.31 | 530 |
| 26 | 4.2 | 55 | 41 | 0.37 | 0.60 | 480* |
| 27 | 4.0 | 59 | 46 | 0.37 | 0.60 | 430* |
| 28 | 3.3 | 21 | 20 | 0.26 | 0.67 | 500** |

*For these devices, the lifetime LD80 was determined from 4000 cd/m².
**For these devices, the lifetime LD80 was determined from 25,000 cd/m².

```
TABLE 3
Structutal formulae of the materials used
  HIL1
  HTM1
  NPB
  ETM1/H4
  Alq₃
  H1
  H2
  H3
  H5
  H6
  SEB1
  LiQ

HTM2
  ETM2
  TEG1
  TER1
  EBM1
  SEG1
```

1-15. (canceled)

16. A compound of the formula (I)
wherein the following applies to the symbols and indices used:
Ar¹ is an aryl or heteroaryl group having 15 to 60 aromatic ring atoms, which is optionally substituted by one or more radicals R;
Ar² is an aryl or heteroaryl group having 6 to 10 aromatic ring atoms, which is optionally substituted by one or more radicals R¹;
Y is on each occurrence, identically or differently, CR² or N; with the proviso that not more than 2 adjacent Y simultaneously correspond to N;
X is on each occurrence, identically or differently, CR³ or N; with the proviso that not more than 2 adjacent X simultaneously correspond to N;
n is either 1, 2, 3 or 4;
R, R¹ and R² are, identically or differently on each occurrence, H, D, F, Cl, Br, I, CHO, N(R⁴)₂, C(═O)R⁴, P(═O)(R⁴)₂, S(═O)R⁴, S(═O)₂R⁴, CR⁴═C(R⁴)₂, CN, NO₂, Si(R⁴)₃, B(OR⁴)₂, OSO₂R⁴, OH, a straight-chain alkyl, alkoxy or thioalkyl group having 1 to 40 C atoms or a branched or cyclic alkyl, alkoxy or thioalkyl group having 3 to 40 C atoms or an alkenyl or alkynyl group having 2 to 40 C atoms, each of which is optionally substituted by one or more radicals R⁴, where one or more non-adjacent CH₂ groups is optionally replaced by R⁴C═CR⁴, C≡C, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴ and where one or more H atoms is optionally replaced by D, F, Cl, Br, I, CN or NO₂, or an aromatic or heteroaromatic ring system having 5 to 60 ring atoms, which may in each case be substituted by one or more non-aromatic radicals R⁴, or an aryloxy or heteroaryloxy group having 5 to 60 aromatic ring atoms, which is optionally substituted by one or more non-aromatic radicals R⁴, or a combination of these systems, where two or more radicals R, R¹ and/or R² is optionally linked to one another and optionally forms a mono- or polycyclic, aliphatic or aromatic ring system;
R³ is, identically or differently on each occurrence, H, D, F, Cl, Br, I, CHO, N(R⁴)₂, C(═O)R⁴, P(—O)(R⁴)₂, S(═O)R⁴, S(═O)₂R⁴, CR⁴═C(R⁴)₂, CN, NO₂, Si(R⁴)₃, B(OR⁴)₂, OSO₂R⁴, OH, a straight-chain alkyl, alkoxy or thioalkyl group having 1 to 40 C atoms or a branched or cyclic alkyl, alkoxy or thioalkyl group having 3 to 40 C atoms or an alkenyl or alkynyl group having 2 to 40 C atoms, each of which is optionally substituted by one or more radicals R⁴, where one or more non-adjacent CH₂ groups is optionally replaced by R⁴C═CR⁴, CC, Si(R⁴)₂, Ge(R⁴)₂, Sn(R⁴)₂, C═O, C═S, C═Se, C═NR⁴, P(═O)(R⁴), SO, SO₂, NR⁴, O, S or CONR⁴ and where one or more H atoms is optionally replaced by D, F, Cl, Br, I, CN or NO₂, or a combination of these systems, where two or more radicals R³ is optionally linked to one another and may form a mono- or polycyclic, aliphatic ring system;
R⁴ is on each occurrence, identically or differently, H, D, F or an aliphatic, aromatic and/or heteroaromatic organic radical having 1 to 20 C atoms, in which, in addition, one or more H atoms is optionally replaced by F; two or more identical or different substituents R⁴ here may also be linked to one another and form a mono- or polycyclic, aliphatic or aromatic ring system,
wherein, in the case where Ar¹ represents a benzo[a]anthracene derivative, this is bonded to the group Ar² in positions 1, 2, 3, 4, 5, 6, 8, 9, 10, 11 or 12.

17. The compound according to claim 16, wherein Ar¹ represents an aryl or heteroaryl group having 18 to 30 aromatic ring atoms, which is optionally substituted by one or more radicals R.

18. The compound according to claim 16, wherein Ar¹ represents an angularly condensed, non-linear aryl group having 18 to 30 aromatic ring atoms, which is optionally substituted by one or more radicals R.

19. The compound according to claim 16, wherein Ar¹ represents a benzo[a]anthracene derivative of the formula (A), which is optionally substituted by one or more radicals R,
where the bond to the group Ar² in formula (I) is optionally localised at positions 1, 2, 3, 4, 5, 6, 8, 9, 10, 11 or 12 of the benzo[a]anthracene skeleton,
or in that Ar¹ represents a benzo[a]phenanthrene derivative of the formula (B), which is optionally substituted by one or more radicals R,
where the bond to the group Ar² in formula (I) is optionally localised at positions 1, 2, 3, 4, 5, 6, 7, 8, 9, 10, 11 or 12 of the benzo[a]phenanthrene skeleton,
or in that Ar¹ represents a benzo[c]phenanthrene derivative of the formula (C), which is optionally substituted by one or more radicals R,
where the bond to the group Ar² in formula (I) is optionally localised at positions 1, 2, 3, 4, 5, 6, 7, 8, 9, 10, 11 or 12 of the benzo[c]phenanthrene skeleton,
or in that Ar¹ represents a benzo[a]phenanthrene derivative of the formula (D), which is optionally substituted by one or more radicals R,
where the bond to the group Ar² in formula (I) is optionally localised at positions 1, 2, 3, 4, 5, 6, 7, 8, 9, 10, 11 or 12 of the benzo[l]phenanthrene skeleton,
or in that Ar¹ represents a benzo[a]pyrene derivative of the formula (E), which is optionally substituted by one or more radicals R,
where the bond to the group Ar² in formula (I) is optionally localised at positions 1, 2, 3, 4, 5, 6, 7, 8, 9, 10, 11 or 12 of the benzo[a]pyrene skeleton,
or in that Ar¹ represents a benzo[e]pyrene derivative of the formula (F), which is optionally substituted by one or more radicals R,
where the bond to the group Ar² in formula (I) is optionally localised at positions 1, 2, 3, 4, 5, 6, 7, 8, 9, 10, 11 or 12 of the benzo[e]pyrene skeleton.

20. The compound according to claim 16, wherein the compound is one of the formulae (I-1a) to (I-6b):
where Z is on each occurrence, identically or differently, CR¹ or N, and the remaining symbols and indices occurring are as defined in claim 16.

21. The compound according to claim 16, wherein n is equal to 1.

22. The compound according to claim 16, wherein 0, 1, 2 or 3 groups Z per aromatic six-membered ring are equal to N and the remaining groups Z are equal to CR¹.

23. The compound according to claim 16, wherein all groups Z are equal to CR¹.

24. The compound according to claim 16, wherein 0, 1 or 2 groups Y per formula are equal to N and all remaining groups Y are equal to CR².

25. The compound according to claim 16, wherein 0, 1, 2 or three groups X are equal to N and the remaining groups are equal to CR³.

26. The compound according to claim 23, wherein all groups X are equal to CR³ and all groups Y are equal to CR²

27. A process for the preparation of the compound according to claim 16, which comprises reacting a compound of the formula (Z-2)
Ar¹-A formula (Z-2)
in an organometallic coupling reaction, with a compound of the formula (Z-3)
A-Ar²-A formula (Z-3)
to give a compound Ar¹—Ar²-A, and the product is subsequently reacted, in a further organometallic coupling reaction, with a compound of the formula (Z-1)
where Ar¹, Ar², X and Y are as defined in claim 16, and A represents any desired reactive group and is preferably selected from I, Br, Cl, F, O-tosylates, O-triflates, O-sulfonates, boric acid, boric acid esters, partially fluorinated silyl groups, diazonium groups and organotin compounds.

28. An oligomer, a polymer or a dendrimer comprising one or more compounds according to claim 16, where the bond(s) to the polymer, oligomer or dendrimer is optionally localised at any desired positions substituted by R, R¹, R² or R³ in formula (I).

29. A formulation comprising at least one compound according to claim 16 and at least one solvent.

30. A formulation comprising at least one polymer, oligomer or dendrimer according to claim 28 and at least one solvent.

31. An electronic device which comprises one or more compounds according to claim 16.

32. An electronic device which comprises at least one polymer, oligomer or dendrimer according to claim 26.

33. The electronic device according to claim 31, wherein the device is an organic electroluminescent device (OLED), organic integrated circuit (O-IC), organic field-effect transistor (O-FET), organic thin-film transistor (O-TFT), organic light-emitting transistor (O-LET), organic solar cell (O-SC), organic optical detector, organic photoreceptor, organic field-quench device (O-FQD), light-emitting electrochemical cell (LEC) or organic laser diode (O-laser).

34. The electronic device according to claim 31, wherein the device is an organic electroluminescent device (OLED).

35. An electronic device which comprises the according to claim 16 and wherein the compound is employed as host material, as fluorescent dopant, as hole-transport material, as hole-injection material or as electron-transport material.
